(19) United States
(12) Patent Application Publication
(10) Pub. No.: US 2005/0159312 A1
(43) Pub. Date: Jul. 21, 2005

(54) Tryptophan aminotransferase, indole-3-pyruvate decarboxylase and indole-3-acetaldehyde oxidase as novel targets for herbicides

(76) Inventors: Klaus Grossmann, Neuhofen (DE); Helmut Schiffer, Grossfischlingen (DE); Matthias Witschel, Bad Durkheim (DE); Cyril Zagar, Mannheim (DE); Costin Rentzea, Heidelberg (DE); Markus Menges, Kassel (DE)

Correspondence Address: NOVAK DRUCE DELUCA & QUIGG, LLP, 1300 EYE STREET NW, SUITE 400 EAST, WASHINGTON, DC, 20005, US

(21) Appl. No.: 10/508,837

(22) PCT Filed: Mar. 19, 2003
(86) PCT No.: PCT/EP03/02846

(30) Foreign Application Priority Data
Mar. 25, 2002 (DE) 102133328

(51) Int. Cl. C12Q 1/48; A01N 25/00; C07C 237/20

(52) U.S. Cl. 504/116.1; 435/15; 504/335; 564/153

(57) ABSTRACT

The present invention relates to tryptophan aminotransferase, indole-3-pyruvate decarboxylase and indole-3-acetaldehyde oxidase as novel targets for herbicides, to test methods for identifying herbicidally active inhibitors of one or more of the abovementioned enzymes, to the herbicidally active inhibitors identified by means of this method, and to methods for controlling undesired vegetation based on the inhibitors according to the invention.

The present invention relates to tryptophan aminotransferase, indole-3-pyruvate decarboxylase and indole-3-acetaldehyde oxidase as novel targets for herbicides, to test methods for identifying herbicidally active inhibitors of one or more of the abovementioned enzymes, to the herbicidally active inhibitors identified by means of this method, and to methods for controlling undesired vegetation based on the inhibitors according to the invention.

In order to find novel herbicides, the candidate active ingredients are applied to suitable test plants following a conventional procedure. The disadvantage of this method is that relatively large amounts of substance are required for testing. Moreover, when the substance is applied directly to the plants to be tested, it must meet extremely high requirements even in the first screening step, since not only the inhibition or other modulation of the activity of the cellular target (as a rule a protein or enzyme as site of action for a herbicide) are required, but the substance must initially reach this target in the first place. Even in this first step, the test substance must meet high requirements with regard to uptake by the plant, permeability across the various cell walls and membranes, persistence for reaching the desired effect, before, finally, an inhibition/modification of the activity of the desired target enzyme may take place.

In view of these requirements, it is not surprising that, firstly, the costs involved in the identification of novel active ingredients spiral and that, secondly, fewer and fewer active ingredients are discovered.

It is an object of the present invention to provide novel targets for herbicides.

Surprisingly, it has been found that the synthesis of indole-3-acetic acid from L-tryptophan is inhibited in vitro in the presence of by the compound of the formula I (see Example 3 and Example 4).

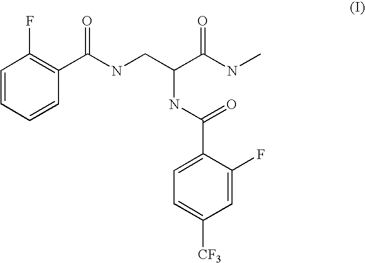

Since compounds of the formula I are herbicidally active (see Example 4), tryptophan aminotransferase, indole-3-pyruvate decarboxylase and indole-3-acetaldehyde oxidase are suitable as targets for herbicides.

We have achieved this object by providing tryptophan aminotransferase, indole-3-pyruvate decarboxylase and indole-3-acetaldehyde oxidase as novel targets for herbicides. Also provided within the present context were methods for identifying herbicidally active substances, which methods are based on using one or more enzymes selected from the group consisting of the enzymes tryptophan aminotransferase, indole-3-pyruvate decarboxylase and indole-3-acetaldehyde oxidase.

The biosynthesis of indole-3-acetic acid (auxin), an important plant hormone, starts with L-tryptophan. The direct precursor of L-tryptophan is indole. The synthetic pathway which starts with L-tryptophan is as follows in all species of higher plants: L-tryptophan is converted to indole-3-pyruvate by tryptophan aminotransferase, and the indole-3-pyruvate is converted to indole-3-acetaldehyde by indole-3-pyruvate decarboxylase. Then, indole-3-acetaldehyde oxidase catalyzes the conversion of the indole-3-acetaldehyde to indole-3-acetic acid, which, in turn, is converted into indole-3-butyric acid by indole-3-butyric acid synthase. The enzymes tryptophan aminotransferase, indole-3-pyruvate decarboxylase and indole-3-acetaldehyde oxidase are generally present in the form of a multi-enzyme complex (Bartel, B. (1997), Ann. Rev. Plant Physiol. Plant Mol. Biol. 48, 51-66; Müller, A., Weiler, E. W., (2000), Planta 211, 855-863). Depending on the test tissue and the test species, other minor biosynthetic pathways have also been described (Normanly, J., Bartel, B. (1999), Current Opinion in Plant Biology 2, 207-213).

To generate a 2,ω-diaminocarboxylic acid compounds of the formula I, a compound of the formula II, 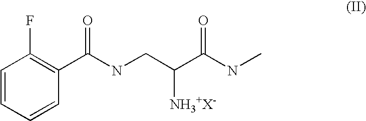
in which A and X^{θ} are a monovalent anion or an anion equivalent, for example Cl^{θ}, Br^{θ} or ½ SO₄^{2θ} of a mineral acid, for example Cl^{θ}, Br^{θ} or ½ SO₄^{2θ}, will be reacted with an aromatic acid halide of the formula III 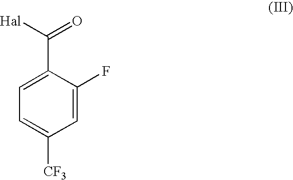
in which Hal is chlorine, bromine or iodine. The aromatic acid halides III are known; in some cases, they are commercially available, or they can be prepared by known methods.

The reaction of the compound II with the compound III is preferably carried out in the presence of a base. The base serves to neutralize the mineral acid H-Hal and H—X formed during the reaction. The base is preferably employed in at least an equimolar amount, in particular in an amount of from 1 to 3 mol per mole of acid H-Hal and H—X to be neutralized.

The reaction of the compound II with the compound III is preferably carried out in a solvent or diluent. Suitable for this purpose are water, diethyl ether, tetrahydrofuran, acetonitrile, ethyl acetate, dichloromethane or toluene.

The reaction temperatures can be varied within a certain range which is predetermined by the stability of the acid chloride III. The process is preferably carried out at temperatures in the range of from 0 to 30° C.

Work-up is carried out by customary methods, for example by treating the reaction mixture with cold water, removing the organic phase and concentrating it after drying under reduced pressure. If required, the residue which remains can be freed from any impurities in the customary manner by chromatography or crystallization.

The compounds of the formula II can be prepared by first reacting, in a first step, a partially protected 2,ω-diaminocarboxylic acid of the formula IV or its acid addition salt, 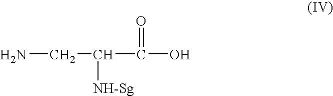
in which Sg is a protective group, with an acid halide of the formula V, 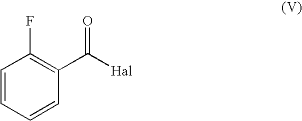
in which Hal is chlorine, bromine or iodine; reacting, in a second step, the resulting compound of the formula 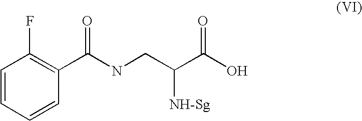
with an amine of the formula CH₃NH₂ in the presence of a suitable condensing agent, and finally removing the protective group Sg.

Suitable protective groups are those which can be eliminated under conditions which do not lead to cleavage of the NH—X bond in the compounds of the formula VI. Suitable protective groups are known from peptide chemistry. They include in particular protective groups which are eliminated by the action of acids with, preferably, an acid strength above acetic acid, for example the tert-butyloxycarbonyl group, the 1-adamantyloxycarbonyl group and the 2-(trimethylsilyl)ethoxycarbonyl group.

Condensing agents for the reaction of compound VI with the amine CH₃NH₂ which are suitable are all reagents which are capable of activating free carboxyl groups, such as propanephosphonic anhydride (PPPA, H. Wissmann et al, Angew. Chem. 92, 129 (1980); H. Wissmann, Phosphorus, Sulfur 30, 645 (1986); M. Feigel, J. Am. Chem. Soc 108, 181 (1986)), N-ethoxycarbonyl-2-ethoxy-1,2-dihydroquinoline (EEDQ, B. Belleau et al, J. Amer. Chem. Soc. 90, 1651 (1968)), diphenylphosphoryl azide (DPPA, Shun-ichi-Yamada et al, J. Am. Chem. Soc. 94, 6203 (1972)) and diethylphosphoryl cyanide (DEPC, Shun-ichi-Yamada et al, Tetrahedron Lett. 18, 1595 (1973)), carbodiimides (Houben-Weyl, Methoden der Organischen Chemie [Methods in organic chemistry], Vol. 15/2, pages 103-115, IVth edition, G. Thieme Verlag), to mention only a few examples of condensing reagents. The reaction conditions described therein can be applied to the reaction according to the invention of compound VI with the amine HNR₂R₃, so that these publications are likewise incorporated by reference.

Removal of the protective group Sg, for example the tert-butoxycarbonyl (BOC) group, from the resulting compounds VII 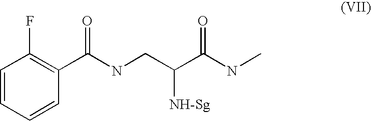
in which Sg has the abovementioned meanings, is generally carried out with an acid, preferably with the aid of trifluoroacetic acid, for example by the methods described by B. Lundt et al, Int. J. Pept. Protein Res., 12, 258 (1978)) or, for example, with 2N hydrogen chloride in dioxane by the methods described by R. Andruszkievicz et al, J. Med. Chem. 30, 1715 (1987)) and yields the abovementioned intermediates of the formula (II) according to the invention in good yields.

The carboxylic acid halides and sulfonyl halides III and V, which are employed as starting compounds, are known or can be prepared by known methods.

2-N-protected 2,ω-diamino acids, such as compound IV, are likewise known, commercially available or can be prepared by known methods, for example by the method of N. Kucharczyk et al, Synth. Commun. 19, 1603 (1989); M. Waki et al, Synthesis, 266 (1981) and Lin-Hua Zhang et al, J. Org. Chem. 62, 6918 (1997).

The methods for identifying compounds with herbicidal activity comprise the following steps:
a) bringing one or more enzymes selected from the group consisting of the enzymes tryptophan aminotransferase, indole-3-pyruvate decarboxylase and indole-3-acetaldehyde oxidase with one or more test substances under conditions which permit the binding of the test substance(s) to one of the abovementioned enzymes or to the nucleic acid sequence which encodes one of the above-mentioned enzymes; and
b) detecting if the test substances reduce or block the transcription, translation or expression of at least one of the abovementioned enzymes; or
c) detecting whether the test substances reduce or block the activity of at least one of the abovementioned enzymes; or
d) detecting whether the test substance binds to one of the abovementioned enzymes.

Under reduction or blocking of the transcription, expression, translation or the activity in step b) and c) is a significant decrease in comparison with the transcription, expression, translation or the activity which is determined in comparison with a method which differs from the abovementioned method by the fact that no test substance is added.

A significant decrease is understood as meaning a decrease of at least 10%, advantageously at least 20%, preferably at least 30%, especially preferably by at least 50% and very especially preferably by at least 70% up to 100%.

The detection in accordance with step b) of the abovementioned method can be carried out with blotting methods with which the skilled worker is familiar.

The detection in accordance with step d) of the above method can be effected using techniques which identify the interaction between protein and ligand. In this context, five preferred embodiments which are also suitable for high-throughput methods (HTS) in connection with the present invention, must be mentioned in particular:
1. The average diffusion rate of a fluorescent molecule as a function of the mass can be determined in a small sample volume via fluorescence correlation spectroscopy (FCS) (Proc. Natl. Acad. Sci. USA (1994) 11753-11575). FCS can be employed for determining protein/ligand interactions by measuring the changes in the mass, or the changed diffusion rate which this entails, of a test compound when binding to the enzyme. An assay system can be designed directly for measuring the binding of a test compound labeled by a fluorescent molecule. As an alternative, the assay system can be designed in such a way that a chemical reference compound which is labeled with a fluorescent molecule is displaced by further test compounds (“displacement assay”). The compounds binding to the enzyme which are identified in this manner are suitable as inhibitors.
2. Fluoresence polarization exploits the characteristic of a quiescent fluorophore excited with polarized light to likewise emit polarized light. If, however, the fluorophore is allowed to rotate during the excited state, the polarization of the fluorescent light which is emitted is more or less lost. Under otherwise identical conditions (for example temperature, viscosity, solvent), the rotation is a function of molecule size, whereby findings regarding the size of the fluorophore-bound residue can be obtained via the signal (Methods in Enzymology 246 (1995), pp. 283-300). An assay system can be designed directly for measuring the binding of a test compound, which is labeled with a fluorescent molecule, to the enzyme. As an alternative, the assay system may also take the form of the “displacement assay” described under 1. The compounds binding to the enzyme which are identified in this manner are suitable as inhibitors.
3. Fluorescent resonance energy transfer (FRET) is based on the irradiation-free energy transfer between two spatially adjacent fluorescent molecules under suitable conditions. A prerequisite is that the emission spectrum of the donor molecule overlaps with the excitation spectrum of the acceptor molecule. By providing the enzyme with a fluorescent label and binding test compounds, the binding can be measured by means of FRET (Cytometry 34, 1998, pp. 159-179). As an alternative, the assay may also take the form of the “displacement assay” described under 1. An especially suitable embodiment of FRET technology is “Homogeneous Time Resolved Fluorescence” (HTRF) as can be obtained from Packard BioScience. The compounds binding to the enzyme which are identified in this manner are suitable as inhibitors.
4. Surface-enhanced laser desorption/ionization (SELDI) in combination with a time-of-flight mass spectrometer (MALDI-TOF) makes possible the rapid analysis of molecules on a support and can be used for analyzing protein/ligand interactions (Worral et al., (1998) Anal. Biochem. 70: 750-756). In a preferred embodiment, the enzyme is immobilized on a suitable support and incubated with the test compound. After one or more suitable wash steps, the test compound molecules which are additionally bound to the enzyme can be detected by means of the abovementioned methodology and inhibitors can thus be selected. The compounds binding to the enzyme which are identified in this manner are suitable as inhibitors.
5. The measurement of surface plasmon resonance is based on the change in the refractive index at a surface when a test compound binds to a protein which is immobilized to said surface. Since the change in the refractive index is identical for virtually all proteins and polypeptides for a defined change in the mass concentration at the surface, this method can be applied to any protein in principle (Lindberg et al. Sensor Actuators 4 (1983) 299-304; Malmquist Nature 361 (1993) 186-187). The measurement can be carried out for example with the automatic analyzer based on surface plasmon resonance which is available from Biacore (Freiburg) at a throughput of, currently, up to 384 samples per day. An assay system can be designed directly for measuring the binding of a test compound to the protein according to the invention. As an alternative, the assay system may also take the form of the “displacement assay” described under 1. The compounds to the enzyme which are identified in this manner are suitable as inhibitors.

All of the substances identified via the above-mentioned methods can subsequently be checked for their herbicidal action in another embodiment of the method according to the invention.

A preferred embodiment of the detection in accordance with step d) comprises treating the test compound
a) with a plant cell lysate which comprises at least one of the enzymes tryptophan aminotransferase, indole-3-pyruvate decarboxylase and indole-3-acetaldehyde oxidase or
b) with at least one of the enzymes tryptophan aminotransferase, indole-3-pyruvate decarboxylase and indole-3-acetaldehyde oxidase which are either partially or fully purified, and
c) the enzymatic activity of at least one of the abovementioned enzymes being subsequently determined in comparison with the activity of at least one of the abovementioned enzymes which has/have not been treated with a test compound, those chemical compounds which reduce or block the activity of at least one of the above-mentioned enzymes being selected.

The partially or fully purified enzymes employed in step b) can be obtained from plant cells by procedures with which the skilled worker is familiar, for example as described in Truelsen, T. A. (Physiol. Plant. 28 (1973) 67-70). In this context, any plant species in which auxin biosynthesis takes place via the metabolic pathway described at the outset may be used for isolating the enzymes. Examples which may be mentioned, but not by limitation, are representatives of the Leguminosae (Fabaceae) family such as mung beans (Vigna radiata), bean (Phaseolus vulgaris) or pea (Pisum sativum) and representatives of the Gramineae (Poaceae) family such as maize (Zea mays). Other representatives of the Leguminosae family are known to the skilled worker and described, for example, in Strasburger “Lehrbuch der Botanik” [Textbook of Botany], Sitte P., Ziegler, H., Ehrendorfer, F., Bresinsky, A., Gustav Fischer, Verlag, Stuttgart.

To identify compounds in step c), the enzymatic activity of the enzyme in question is determined after a reaction time in comparison with the activity of the non-inhibited enzyme. In this way, compounds which result in a significant decrease in the enzymatic activity are selected.

“Reaction time” refers to the time required for carrying out an activity assay until a significant finding regarding an activity is obtained. It depends both on the specific activity of the protein employed in the assay and on the method used and the sensitivity of the apparatus used. The skilled worker is familiar with the determination of the reaction times. In the case of assay systems which are based on photometry, the reaction times are in general between >0 to 120 minutes.

The activity can be determined by incubating the enzyme in question with a suitable substrate, the conversion rate of the substrate or the increase in the resulting product being monitored spectroscopically.

Examples of substances which may be employed as substrate are tryptophan, indole-3-pyruvate and indole-3-acetaldehyde. In addition, radioactive derivatives or tryptophan, indole-3-pyruvate and indole-4-acetaldehyde derivatives modified with chromophoric or fluorophoric groups, which make possible, for example, a spectroscopic determination, may also be employed in the methods according to the present invention.

Depending on the substrate employed, there are, in principle, three different methods for determining the enzymatic activity:

Variant 1 a)-e):

If tryptophan is employed as substrate for determining the enzymatic activity of at least one of the enzymes selected from the group consisting of the enzymes tryptophan aminotransferase, indole-3-pyruvate decarboxylase and indole-3-acetaldehyde oxidase in step c) of method I, the enzymatic activity can be determined via
a) the decrease in L-tryptophan; or
b) the increase in indole-3-pyruvate; or
c) the increase in indole-3-acetaldehyde; or
d) the increase in indole-3-acetic acid; or
e) a combination of at least two of methods (a) to (d).
   Variant 2 a)-d):

If indole-3-pyruvate is employed as substrate for determining the enzymatic activity of one or both of the enzymes selected from the group consisting of the enzymes indole-3-pyruvate decarboxylase and indole-3-acetaldehyde oxidase in step c) of method I, the enzymatic activity can be determined via
a) the decrease in indole-3-pyruvate; or
b) the increase in indole-3-acetaldehyde; or
c) the increase in indole-3-acetic acid; or
d) a combination of at least two of the methods (a) to (c).
   Variant 3 a)-c):

If indole-3-acetaldehyde is used as substrate for determining the enzymatic activity of indole-3-acetaldehyde oxidase in step (c) of process I, the enzymatic activity can be determined via
a) the decrease in indole-3-acetaldehyde; or
b) the increase in indole-3-acetic acid; or
c) a combination of methods (a) and (b).

Here, the choice of the process variant, but also the combinations of methods mentioned in the variants in question, depend on the composition of the enzyme solution employed in the assay and/or on the enzymatic activity to be determined.

If the enzyme solution employed for the assay comprises the enzymes tryptophan aminotransferase, indole-3-pyruvate decarboxylase and indole-3-acetaldehyde oxidase, the following methods are possible: 1 a), 1 d), 1 e) (e=a+d), 2 a), 2 c), 2 d) (d=a+c) and 3 a), 3 b), 3 c) (c=a+b).

If the enzyme solution employed in the assay comprises tryptophan aminotransferase and indole-3-pyruvate decarboxylase, the following methods are possible: 1 a), 1 c), 1 e) (e=a+c), 2 a), 2 b), 2 d) (d=a+b).

If the enzyme solution employed in the assay comprises indole-3-pyruvate decarboxylase and indole-3-acetaldehyde oxidase, the following methods are possible: 2 a), 2 c), 2 d) (d=a+c) and 3 a), 3 b), 3 c) (c=a+b).

If the enzyme solution employed in the assay comprises tryptophan aminotransferase, the following methods are possible: 1 a), 1 b), 1 e) (e=a+b).

If the enzyme solution employed in the assay comprises indole-3-pyruvate decarboxylase, the following methods are possible: 2 a), 2 b), 2 d) (d=a+b).

If the enzyme solution employed in the assay comprises indole-3-acetaldehyde oxidase, the following methods are possible: 3 a), 3 b), 3 c) (c=a+b).

In a preferred embodiment of the variants mentioned above, a mixture of the three enzymes tryptophan aminotransferase, indole-3-pyruvate decarboxylase and indole-3-acetaldehyde oxidase is used for determining the enzymatic activity, which, as mentioned above, makes possible the following assay methods: 1 a), 1 d), 1 e) (e=a+d), 2 a), 2 c), 2 d) (d=a+c) and 3 a), 3 b), 3 c) (c=a+b).

Suitable spectroscopic methods for the above-mentioned methods 1, 2 and 3 are mass spectroscopy or UV-VIS spectroscopy, LC/UV-VIS (liquid chromatography/UV-VIS spectroscopy) or UV-VIS spectroscopy. Particularly suitable for each of the variants 1b), 1d), i e), 2 d) and 3 c) are mass spectroscopy such as LC/MS or HPLC/MS or various LC- or HPLC-based methods in conjunction with conductivity measurements, NMR measurements or refractive index measurements, since it makes possible the simultaneous determination of different substances.

In a preferred embodiment, the photometric determination follows a method described by Simpson, R. M. et al. (Planta 201 (1997) 71-77) and Truelsen, T. A. et al. (Phsiol. Plant. (1972) 26, 289-295), the determination by mass spectroscopy follows a method described by Simpson, R. M. et al. (Planta 201 (1997), 71-77).

In an especially preferred embodiment, the increase or decrease of tryptophan and/or indole-3-pyruvate and/or increase in indole-3-acetic acid and/or indole-3-butyric acid for determining the enzymatic activity is determined photometrically.

The photometric determination can be detected for example by the increase or decrease in indole-3-pyruvate at a wavelength λ=328 nm (Simpson, R. M., Nonhebel, H. M., Christie, D. L. (1997), Planta 201, 71-77, Truelsen, T. A. (1972), Phsiol. Plant., 26, 289-295).

The increase or decrease in indole-3-acetic acid can be detected by means of fluorescence (Ex=254 nm; Em=360 nm; method of Mazur H. et al, J. Appl. Phycology 13 (2001) 35-42).

In addition, all of the enzyme inhibitors identified in the abovementioned method can be tested for their herbicidal activity in an in-vivo activity test. Here, the substance in question is applied to the harmful plant in question in order to test the herbicidal activity.

All of the abovementioned methods are hereinbelow referred to under “method according to the invention”.

The method according to the invention can be carried out in individual separate set-ups and/or, advantageously, jointly or particularly advantageously in a high-throughput screen set-up and thus used for identifying compounds with herbicidal activity.

It is also possible to employ several test compounds in the method according to the invention. If the target is affected by a group of test compounds, then it is either possible directly to isolate the individual test compounds or to divide the group of test compounds into various subgroups, for example when it consists of a multiplicity of different components, and thus reducing the number of different test compounds in the assay system. The method according to the invention is then repeated with the individual test compound or the corresponding subgroup of the test compounds. Depending on the complexity of the sample, the abovedescribed steps can be repeated several times, preferably until the subgroup identified in accordance with the method according to the invention only comprises a small number of test compounds, or indeed only one test compound.

The invention furthermore relates to compounds identified by the methods according to the invention. These compounds are hereinbelow referred to as “selected compounds”. They have a molecular weight of less than 1 000 g/mol, preferably less than 900 g/mol, preferably less than 800 g/mol, especially preferably less than 700 g/mol, very especially preferably less than 600 g/mol, and a Ki value of less than 1 mM.

The compounds identified by the methods according to the invention may of course also be present in the form of their agriculturally useful salts.

Suitable among the agriculturally useful salts are mainly the salts of those cations, or the acid addition salts of those acids, whose cations, or anions, respectively, do not adversely affect the herbicidal activity of the compounds with herbicidal activity which have been identified by the methods according to the invention.

Furthermore, if the selected compounds comprise asymmetrically substituted α-carbon atoms, they can be present in the form of racemates, enantiomer mixtures, pure enantiomers or, if they comprise chiral substituents, also as diastereomer mixtures.

The selected compounds can be substances which have been prepared by chemical synthesis or produced by microorganisms and they can be found, for example, in cell extracts of, for example, plants, animals or microorganisms. The reaction mixture can be a cell-free extract or comprise a cell or cell culture. Suitable methods are known to the skilled worker and are described in general terms in, for example, Alberts, Molecular Biology the cell, 3^{rd} Edition (1994), for example chapter 17.

Possible test compounds can be expression libraries such as, for example, cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein).

The selected compounds can be used for controlling undesired vegetation and, under certain circumstances, also for the defoliation, for example of potatoes, or for the desiccation of, for example, cotton. Moreover, the selected compounds may, if appropriate, also be used for regulating the growth of plants since inhibitors of the biosynthesis of the plant hormone auxins may have an effect on the growth of the plants. Agrochemical compositions which comprise the selected compounds effect very good control of vegetation on non-crop areas, especially at high application rates. In crops such as wheat, rice, maize, soya and cotton, they act against broad-leaved weeds and grass weeds without inflicting any major damage on the crop plants. This effect is especially observed at low application rates. The selected compounds can be used for controlling the harmful plants which have already been mentioned above.

Depending on the application method in question, selected compounds, or agrochemical compositions comprising them, can advantageously also be employed in a further number of crop plants for eliminating undesired plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the selected compounds can also be used in crops which tolerate the effect of herbicides owing to breeding, including recombinant methods.

The invention furthermore relates to a method for preparing the agrochemical composition which has already been mentioned above, which comprises formulating selected compounds with auxiliaries which are suitable for the art of formulation.

The selected compounds can be formulated for example in the form of directly sprayable aqueous solutions, powders, suspensions, also highly concentrated aqueous, oily or other suspensions or suspoemulsions or dispersions, emulsifiable concentrates, emulsions, oil dispersions, pastes, dusts, materials for broadcasting or granules, and applied by spraying, atomizing, dusting, spreading or pouring. The use forms depend on the intended purpose and on the nature of the selected compounds; in any case, they should ensure as fine as possible a distribution of the selected compounds. The agrochemical composition comprise a herbicidally active amount of at least one selected compound and auxiliaries conventionally used for formulating agrochemical compositions.

For the preparation of emulsions, pastes or aqueous or oil-comprising formulations and of dispersible concentrates (DC), the selected compounds can be dissolved or dispersed in an oil or solvent, it being possible to add further formulation auxiliaries for the purposes of homogenization. However, it is also possible to prepare liquid or solid concentrates which consist of selected compound and, if appropriate, solvents or oil and optionally further auxiliaries, such concentrates being suitable for dilution with water. To be mentioned are: emulsifiable concentrates (EC, EW), suspensions (SC), soluble concentrates (SL), dispersible concentrates (DC), pastes, pills, wettable powders or granules, it being possible for the solid formulations either to be soluble or dispersible (wettable) in water. Moreover, suitable powders, granules or tablets may additionally be provided with a solid coating which prevents abrasion or early release of active compounds.

In principle, the term auxiliaries is understood as meaning the following classes of substance: defoamers, thickeners, wetters, stickers, dispersants, emulsifiers, bactericides and/or thixotropes. The meaning of the abovementioned agents is known to the skilled worker.

SLs, EWs and ECs can be prepared by simply mixing the constituents in question, while powders are prepared by mixing or grinding in specific types of mills (for example hammer mills). DC, SCs and SEs are conventionally prepared by wet milling, it being possible to prepare an SE from an SC by addition of an organic phase which may comprise further auxiliaries or selected compounds. The preparation is known. Powders, materials for spreading and dusts can advantageously be prepared by mixing or concomitantly grinding the active substances together with a solid carrier. Granules, for example coated granules, impregnated granules and homogeneous granules, can be prepared by binding the selected compounds to solid carriers. Further preparation details are known to the skilled worker and mentioned for example in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

The skilled worker is familiar with a multiplicity of inert liquid and/or solid carriers which are suitable for the formulations according to the invention, such as, for example, liquid additives such as mineral oil fractions of medium to high boiling point, such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone, or strongly polar solvents, for example amines such as N-methylpyrrolidone or water.

Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and vegetable products such as cereal meal, tree bark meal, wood meal and nutshell meal, cellulose powders or other solid carriers.

The skilled worker is familiar with a multiplicity of surface-active substances (surfactants) which are suitable for the formulations according to the invention, such as, for example, alkali metal, alkaline earth metal and ammonium salts of aromatic sulfonic acids, for example lignosulfonic acid, phenolsulfonic acid, naphthalenesulfonic acid and dibutylnaphthalenesulfonic acid, and of fatty acids, alkylsulfonates and alkylarylsulfonates, alkyl sulfates, lauryl ether sulfates and fatty alcohol sulfates, and salts of sulfated hexa-, hepta- and octadecanols and of fatty alcohol glycol ethers; condensates of sulfonated naphthalene and its derivatives with formaldehyde, condensates of naphthalene or of the naphthalenesulfonic acids with phenol and formaldehyde, polyoxyethylene octylphenol ether, ethoxylated isooctylphenol, octylphenol or nonylphenol, alkylphenyl polyglycol ethers, tributylphenyl polyglycol ether, alkylaryl polyether alcohols, isotridecyl alcohol, fatty alcohol/ethylene oxide condensates, ethoxylated castor oil, polyoxyethylene alkyl ether or polyoxypropylene alkyl ether, lauryl alcohol polyglycol ether acetate, sorbitol esters, lignin-sulfite waste liquors or methylcellulose.

The agrochemical compositions or the selected compounds can be applied pre- or post-emergence. If the selected compounds are less well tolerated by certain crop plants, application techniques may be employed in which the selected compounds are sprayed, with the aid of the spraying apparatus, that they come into as little contact, if any, with the leaves of the sensitive crop plants while the selected compounds reach the leaves of undesired plants which grow underneath, or the bare soil surface (post-directed, lay-by).

The application rates of selected compounds are from 0.001 to 3.0, preferably 0.01 to 1.0 kg/ha, depending on the intended aim of the control measures, the season, the target plants and the growth stage.

The invention is illustrated in greater detail by the following, nonlimiting examples.

### EXAMPLE 1

### Enzyme Isolation of Tryptophan Aminotransferase (TATase) From Etiolated Mung Beans (Method of Simpson, R. M., Nonhebel, H. M., Christie, D. L. (1997), Planta 201, 71-77)

The mung beans (Vigna radiata) are grown for 6-7 days in vermiculite in the dark. At the time of harvesting, the plants are approx. 8 cm in height.

All of the following steps are carried out at 4° C. 100 g of freshly harvested plant material is homogenized in a mixer at 4° C. with 100 ml of extraction buffer (50 mM KH₂PO₄, pH 8.5; 0.5 mM EDTA; 0.5 mM MnCl₂; 10 mM isoascorbate) in 2 steps at 1 minute each. Thereafter, 5 g of polyvinylpolypyrrolidone are added (ratio plant material/PVPP 100:5), the mixture is stirred for 5 minutes and then filtered through 8 layers of gauze. After centrifugation of the filtrate (20 000 g, 4° C. 20 min; SL-250 T rotor—Sorvall Super T 1 centrifuge), the supernatant is to a fractional precipitation with ammonium sulfate (60%, 80%). The supernatant which remains is discarded, the pellet is resuspended with 3 ml of column buffer (10 mM Tris-HCl, pH 8.0; 0.1 M NaCl), the suspension is brought to a total volume of 5.0 ml and the mixture is desalted using an equilibrated PD 10 column (Amersham Pharmacia). Depending on the use, the desalted enzyme solution is either frozen at −20° C. in portions or, if required, can be employed directly in the enzyme assay.

### EXAMPLE 2

### Enzyme Assay

500 μl of assay buffer (0.1 M borax, pH 8.5; 10 mM tryptophan, 0.5 mM sodium arsenate, 0.5 mM EDTA, 50 μM pyridoxal phosphate), 100 μl of a freshly prepared 10 mM α-ketoglutarate solution and approximately 50-250 μl of enzyme solution (5 mg/ml) are employed in a suitable vessel. The assay is made up to 1 ml with water and 10 μl of active ingredient solution, where the added quantity of active ingredient solution should amount to between 1-5% of the total volume of the mixture and not exceed 1 mM, and the mixture is incubated for 30 minutes at 60° C. with shaking. The reaction is subsequently stopped with 150 μl of methanol and the sample is centrifuged for 5 minutes at 20 000 g. The supernatant is analyzed by LCMS, the following equipment/parameters being used (by stating the type of apparatus, an indication of all further parameters should be unnecessary):

Apparatus:

pumps Bio-Tek 522 and 520
column thermostat Bio-Tek 582
autosampler Bio-Tek 560
UV detector Bio-Tek 535
mass detector Applied Biosystems Mariner
   Column:
Phenomenex Aqua 3μ C18 125 A, 150×2 μm
flow rate: 0.3 ml/min
mobile phase:
   buffer A: 0.2% acetic acid
   buffer B: methanol
      Power gradient:
0-30 min 75% to 50% linear buffer A
30-35 min 50% buffer A
35-40 min 50% to 75% linear buffer A
40-45 min 75% buffer A
   UV detector:
280 nm

The retention times required for the evaluation are compiled in table 1.

TABLE 1

| Compound | Retention times: |
|---|---|
| L-Tryptophan | 3.8 min |
| Indole-3-acetic | 23.7 min |
| acid | |
| Indole-3-pyruvate | 35.2 min |
| | 42.5 min *) |

*) This indicates keto-enol tautomerism

### EXAMPLE 3

### Herbicidal Activity of Compound I

The herbicidal activity of the compound I was demonstrated by greenhouse experiments:

The culture containers used were plastic pots comprising loamy sand with approximately 3.0% humus as substrate. The seeds of the test plants were sown separately for each species.

For the pre-emergence treatment, the active ingredients, suspended or emulsified in water, were applied directly after sowing by means of finely distributed nozzles. The containers were irrigated gently to promote germination and growth, and subsequently covered with transparent plastic hoods until the plants had rooted. This cover causes uniform germination of the test plants unless this was adversely affected by the active ingredients.

For the post-emergence treatment, the test plants were first grown to a plant height of from 3 to 15 cm, depending on the plant habit, and only then treated with the active ingredients which had been suspended or emulsified in water. To this end, the test plants were either sown directly and grown in the same containers, or they were first grown separately as seedlings and transplanted into the test containers a few days prior to treatment. The application rate for the post-emergence treatment was 3 kg of a.s./ha.

Depending on the species, the plants were kept at from 10-25° C. or 20-35° C., respectively. The test period extended over 2 to 4 weeks. During this time, the plants were attended, and their response to the individual treatments was evaluated.

Evaluation was carried out using a scale of from 0 to 100. 100 means no emergence of the plants, or complete destruction of at least the aerial parts, while 0 means no damage, or normal course of growth.

The plants used in the greenhouse experiments belong to the following species:

| Scientific name | Common name |
|---|---|
| Chenopodium album | common lambsquarters |
| Echinocloa crusgalli | cockspur(grass) |
| Pharbitis purpurea | morningglory, common |

At application rates of 1.0 kg a.s./ha, compound I was very good herbicidal efficiency post-emergence against Chenopodium album, Echinocloa crus-galli and Pharbitis purpurea.

### EXAMPLE 4

### Enzyme Assay for the Search for Inhibitors of Indole-3-acetic Acid (IEA, Auxin) Biosynthesis

To obtain the enzyme extract, 2 g of frozen plant shoots of Chenopodium album (common lambsquarters), which had been comminuted in a pestle and mortar under liquid nitrogen, were defrosted at room temperature in 3 ml of 100 mM EPPS extraction buffer (5 mM dithiothreitol, 6 μM pyridoxal phosphate, 10 μM leupeptin, 10 μM Pefabloc SC, pH 8.5) together with a spatula-tipfull of polyvinyl pyrrolidone in the course of 30 minutes with gentle stirring. Thereafter, the plant material was centrifuged for 10 minutes at 4° C. and the supernatant was desalinified on a Sephadex G-25 column (preequilibrated with 5 mM EPPS elution buffer (1 mM dithiothreitol, 6 μM pyridoxal phosphate, 10 μM Pefabloc SC, pH 8.5). The resulting enzyme extract (400 μl) was incubated in the test assay (volume 600 μl) for 2 hours at 37° C. in the presence of 20 μM pyridoxal phosphate, 80 mM EPPS extraction buffer, 50 μM L-tryptophan, 50 μM α-ketoglutarate and 50 μM indole with addition of 100 μl of EPPS extraction buffer and 6 μl of active ingredient solution (10 mM compound I in DMSO). The control without compound I was treated with the corresponding amount of DMSO. The reaction was subsequently stopped by addition of 20 μl 7.2N HCl and 3 ml of ethyl acetate. The control assays without incubation were placed on ice and immediately treated with HCl and ethyl acetate. After reextraction with 3 ml of ethyl acetate, the organic phases (6 ml) are combined and concentrated under a nitrogen stream, and the samples are methylated with diazomethane. The indole-3-acetic acid which is present in the sample material was thus converted into methyl indole-3-acetate (IEA-Me). Thereafter, the amount of IEA-Me was determined immunoanalytically by means of monoclonal antibodies (100% reactivity to IEA-Me); (method of Weiler E. W., Eberle J., Mertens R., Atzorn R., Feyerabend M., Jourdan P. S., Arnscheidt A., Wieczorek U., in: Immunology in Plant Science (Wang T. L., Ed.), Society for Experimental Biology, Seminar Series 29, Cambridge University Press, Cambridge, 1986, pp. 27-58) and used as measurement for the enzyme activities. Inhibition of the indole-3-acetic acid (IEA) synthesis reaction by compound 1 is shown in Table 2.

TABLE 2

| | pMol IEA-Me/g fresh | |
|---|---|---|
| | shoot weight² | Inhibition [%]1 |
| Control without | 48.7 +/− 4.5 | |
| incubation | | |
| Control with | 445.3 +/− 33.9 | 0 |
| incubation | | |
| Assay with | 156.0 +/− 14.8 | 73 |
| incubation and | | |
| 100 μM compound I | | |

¹To calculate the % inhibition, the control value without incubation is subtracted from the values.
²Mean from three measurements

1. The use of one or more enzymes selected from the group consisting of the enzymes tryptophan aminotransferase, indole-3-pyruvate decarboxylase and indole-3-acetaldehyde oxidase in a method for identifying compounds with herbicidal activity.

2. A method for identifying herbicidally active substances, comprising the following steps:
a) bringing one or more enzymes selected from the group consisting of the enzymes tryptophan aminotransferase, indole-3-pyruvate decarboxylase and indole-3-acetaldehyde oxidase into contact with one or more test substances under conditions which permit the binding of the test substance(s) to one of the abovementioned enzymes or to the nucleic acid sequence which encodes one of the abovementioned enzymes; and
b) detecting if the test substances reduce or block the transcription, translation or expression of at least one of the abovementioned enzymes; or
c) detecting whether the test substances reduce or block the activity of at least one of the abovementioned enzymes; or
d) detecting whether the test substance binds to one of the abovementioned enzymes.

3. A method as claimed in claim 1, wherein the test compound
a) is treated with a plant cell lysate which comprises at least one of the enzymes tryptophan aminotransferase, indole-3-pyruvate decarboxylase and indole-3-acetaldehyde oxidase or
b) with at least one of the enzymes tryptophan aminotransferase, indole-3-pyruvate decarboxylase and indole-3-acetaldehyde oxidase which are either partially or fully purified, and
c) the enzymatic activity of at least one of the abovementioned enzymes is subsequently determined in comparison with the activity of at least one of the abovementioned enzymes which has/have not been treated with a test compound, those chemical compounds which reduce or block the activity of at least one of the abovementioned enzymes being selected.

4. A method as claimed in claim 2 or 3, wherein tryptophan aminotransferase is employed as the enzyme.

5. A method as claimed in claim 2, wherein tryptophan or a tryptophan derivative is employed as substrate and the enzymatic activity in step (c) is determined via
a) the decrease in L-tryptophan; or
b) the increase in indole-3-pyruvate; or
c) the increase in indole-3-acetaldehyde; or
d) the increase in indole-3-acetic acid; or
e) the increase in Indole-3-butyric acid; or
a combination of at least two of the methods (a) to (e).

6. A method as claimed in claim 2, wherein indole-3-pyruvate or an indole-3-pyruvate derivative is employed as the substrate and the enzymatic activity in step (c) is determined via
a) the decrease in indole-3-pyruvate; or
b) the increase in indole-3-acetaldehyde; or
c) the increase in indole-3-acetic acid; or
d) the increase in indole-3-butyric acid; or
e) a combination of at least two of the methods (a) to (d).

7. A method as claimed in claim 2, wherein indole-3-acetaldehyde or an indole-3-acetaldehyde derivative is employed as the substrate and the enzymatic activity in step (c) is determined via
a) the decrease in indole-3-acetaldehyde; or
b) the increase in indole-3-acetic acid; or
c) a combination of methods a) and b).

8. A method as claimed in one of claims 2 to 7, wherein the enzymatic activity is determined spectroscopically.

9. A method as claimed in any of claims 2 to 8, wherein the substances are identified in the form of a high-throughput-screening.

10. A method as claimed in any of claims 2 to 9, wherein the compound selected by means of the method is applied to a plant to verify the herbicidal activity.

11. The use of compounds with herbicidal or growth-regulatory activity for controlling undesired vegetation, wherein the compounds inhibit one or more compounds selected from the group consisting of the enzymes tryptophan aminotransferase, indole-3-pyruvate decarboxylase and indole-3-acetaldehyde oxidase.

12. The use as claimed in claim 11, wherein the compounds with herbicidal or growth-regulatory activity are formulated with the aid of adjuvents which are suitable for the formulation of agricultural compositions.

13. The compound of the formula (I)

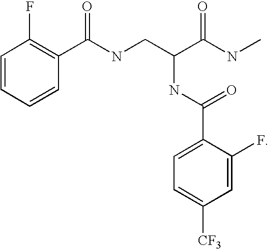

14. The use of compounds as claimed in claim 13 for controlling undesired vegetation.
